(19) United States
(12) Patent Application Publication
(10) Pub. No.: US 2015/0129041 A1
(43) Pub. Date: May 14, 2015

(54) INOCULUM TRANSFER SYSTEM

(71) Applicant: Dow AgroSciences LLC, Indianapolis, IN (US)

(72) Inventor: Chris Stowers, Carmel, IN (US)

(21) Appl. No.: 14/532,140

(22) Filed: Nov. 4, 2014

Related U.S. Application Data
(60) Provisional application No. 61/902,957, filed on Nov. 12, 2013.

(51) Int. Cl. F17D 1/02; A61L 2/07

(52) U.S. Cl. CPC F17D 1/02; A61L 2/07
USPC 137/1; 422/26; 138/106; 137/590

(57) ABSTRACT

A cap for a vessel has a first side and a second side opposite the first side. A first fluid passageway extends from the first side to the second side through the cap; and a second fluid passageway also extends from the first side to the second side through the cap. At least a portion of the second fluid passageway surrounds a portion of the first fluid passageway.

### CROSS-REFERENCE TO RELATED APPLICATIONS

This application claims the benefit of U.S. Provisional Patent Application Ser. No. 61/902,957, filed Nov. 12, 2013, the disclosure of which is expressly incorporated by reference herein

### FIELD

The present invention relates to methods and apparatus for transferring a sample between containers and in particular to methods and apparatus transferring a culture into or out of a fermentation container or vessel.

### BACKGROUND AND SUMMARY

Inoculum transfer systems are used in fermentation to transfer actively growing cultures from one fermenter to another to continue propagation. The inoculum is the portion of the cellular culture transferred to a fermentation vessel. The transfer of inoculum facilitates a scale-up in the volume of culture, and may also be used to ensure that production facilities run continuously.

In one embodiment, a cap for a vessel is provided. The cap has a first side and a second side opposite the first side. A first fluid passageway extends from the first side to the second side through the cap; and a second fluid passageway also extends from the first side to the second side through the cap. At least a portion of the second fluid passageway surrounds a portion of the first fluid passageway.

In one embodiment, a vessel for a flowable material is provided. The vessel has an interior bounded by a top, a bottom, and at least one wall and an aperture in the vessel providing access to the interior. A cap couples to the vessel to cover the aperture. The cap has a first side facing away from the interior of the vessel and a second side facing the interior of the vessel. A first fluid connection through the cap is configured to fluidly couple a first external line to the interior of the vessel. A second fluid connection through the cap is configured to fluidly couple a second external line to the interior of the vessel separately from the first fluid connection. A portion of the second fluid connection surrounds a portion of the first fluid connection.

In one embodiment, a method for transferring a flowable material from a first vessel to a second vessel is provided. The flowable material is provided in an interior of the first vessel. The first vessel includes a cap covering an aperture. The cap has a first fluid passageway and a second fluid passageway through the cap, the first fluid passageway including a tube extending from the cap into the flowable material, at least a portion of the second fluid passageway surrounding the tube. A fluid connection is formed between the first vessel and the second vessel by fluidly connecting an exit of the first fluid passageway and the second fluid passageway with the interior of the second vessel. A pressure is applied to the flowable material in the first vessel, the pressure forcing a portion of the flowable material through the tube and first fluid connection. A portion of the flowable material is received in the interior of the second vessel.

The above mentioned and other features of the invention, and the manner of attaining them, will become more apparent and the invention itself will be better understood by reference to the following description of embodiments of the invention taken in conjunction with the accompanying drawings.

### BRIEF DESCRIPTION OF THE DRAWINGS

FIG. 1 illustrates a fermentation vessel including a dual-port cap connection;

FIG. 2 illustrates a bottom perspective view of the dual-port cap connection of FIG. 1;

FIG. 3 illustrates a sectional view of the dual-port cap connection of FIG. 2;

FIG. 4 is a flow chart of an exemplary method of sterilizing the dual-port cap connection of FIG. 1;

FIG. 5 illustrates the fermentation vessel of FIG. 1 coupled to a second fermentation vessel;

FIG. 6 is a flow chart of an exemplary method of transferring material between the vessels of FIG. 5; and

FIG. 7 illustrates withdrawing and returning a material through the same aperture of a vessel for treatment.

### DETAILED DESCRIPTION OF THE DRAWINGS

The embodiments disclosed below are not intended to be exhaustive or to limit the invention to the precise forms disclosed in the following detailed description. Rather, the embodiments are chosen and described so that others skilled in the art may utilize their teachings. While the present disclosure is primarily directed to transferring inoculum or cultures between fermentation vessels, it should be understood that the features disclosed herein may have application to the transfer of other flowable samples between vessels, or the removal and return of a flowable sample from the same vessel through a single aperture.

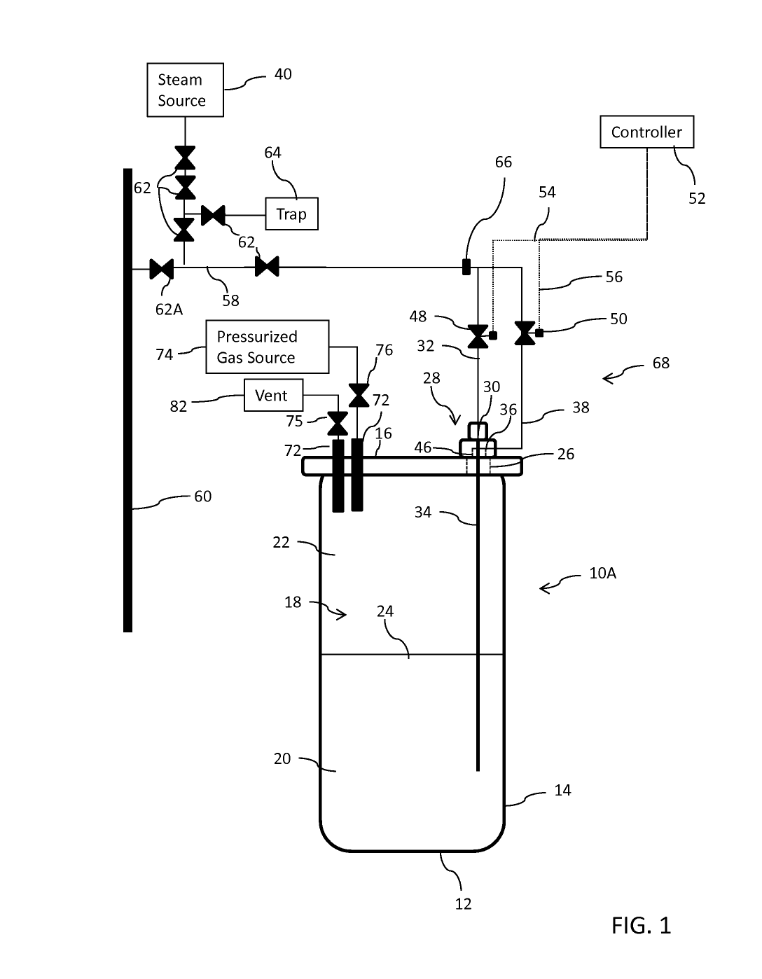

Referring first to FIG. 1, an exemplary vessel 10A is illustrated. Vessel 10A may be, for example, a fermenter, such as a Sterilize-In-Place (“SIP”) fermenter. In a typical SIP fermenter, a nutrient media is added to the interior 18 of the vessel 10A followed by sterilization of the interior 18 and nutrient media by adding sufficient steam to the interior 18 to raise the temperature of the interior 18 and nutrient media for a predetermined time to sterilize the vessel. The vessel 10A includes a bottom 12, at least one wall 14, and a top, illustratively removable headplate 16, defining an interior 18 of the vessel 10A. As shown in FIG. 1, the interior 18 may contain a liquid or flowable material 20 and a headspace 22 containing a gas or vapor located above the surface 24 of the liquid or flowable sample. The vessel 10A may include a vessel jacket (now shown) surrounding the vessel 10A for heating and/or cooling the vessel. The vessel jacket illustratively contains water, steam, a heat transfer agent such as glycol, or a combination thereof.

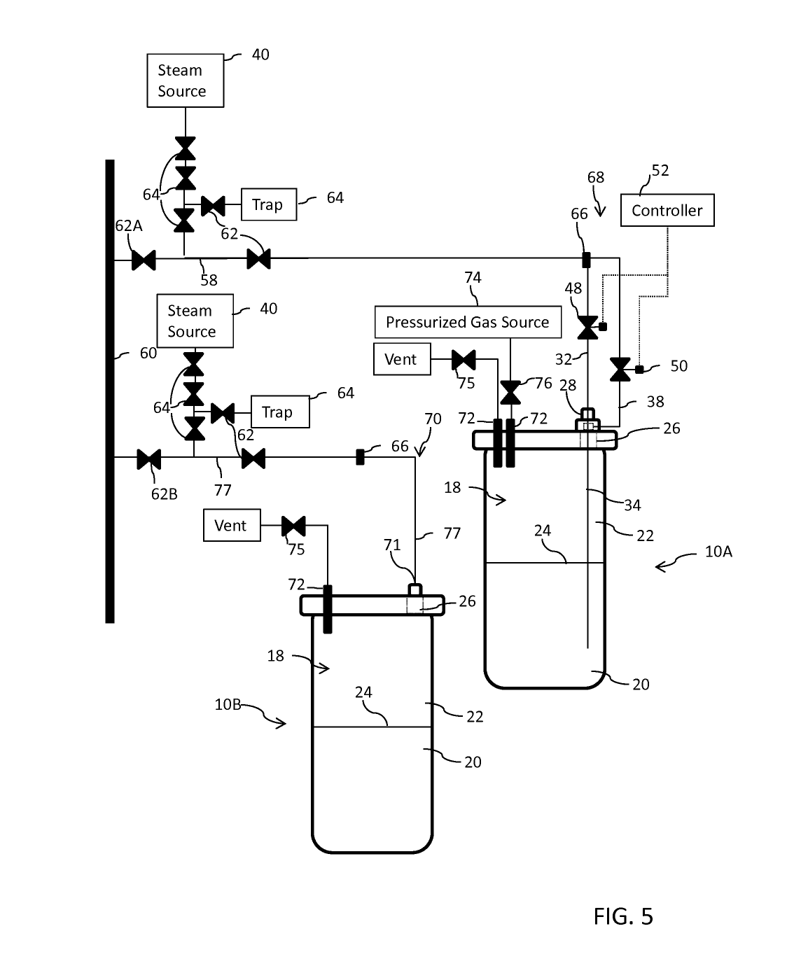

In one embodiment, the liquid or material 20 is a cell culture. Exemplary cell cultures include a culture in an aqueous medium, and may further include nutrient media and suitable additives. In one embodiment, vessel 10A provides a sterile environment in which the cell culture may be grown and reproduced. Vessel 10A may be attached to additional vessels, such as vessel 10B (See FIG. 5), through one or more lines, such as inoculum subheader 60, that can be sterilized between uses to prevent contamination of the cell culture being transferred.

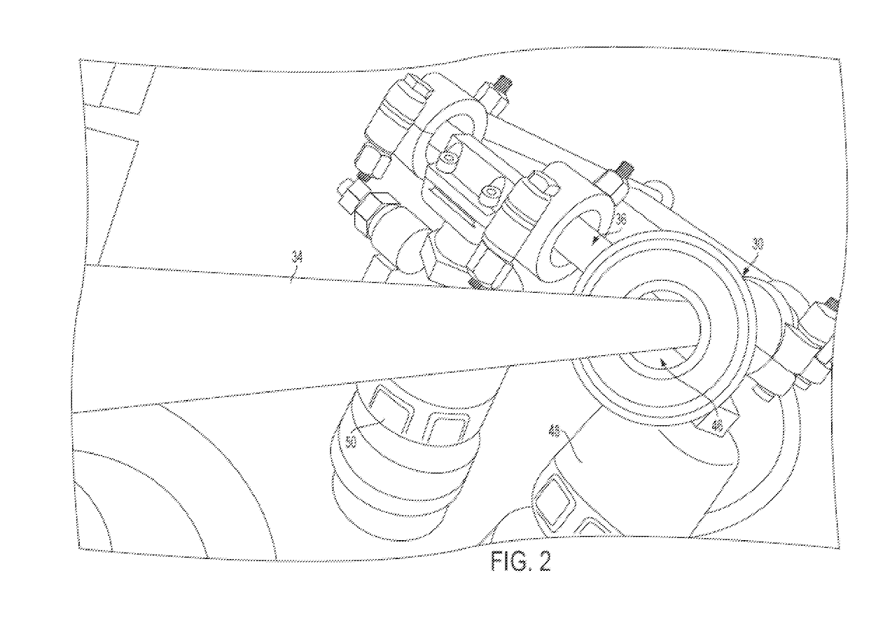

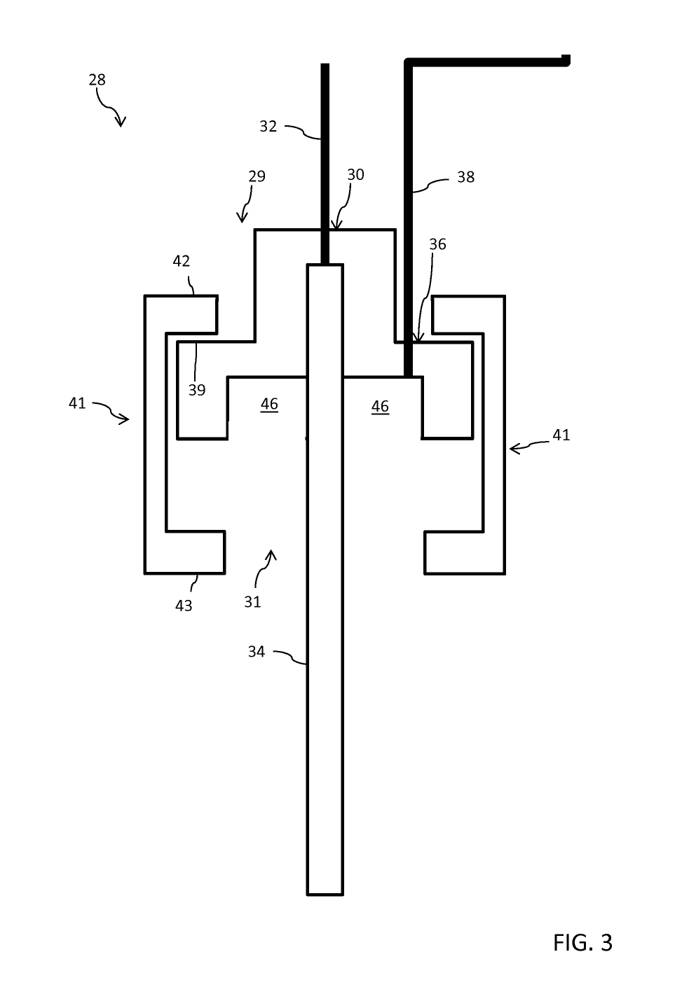

In one embodiment, vessel 10A includes an aperture 26 through headplate 16 providing access to the interior 18. In one embodiment, vessel 10A includes dual-port cap 28 providing access to aperture 26. As described in more detail below, dual-port cap 28 provides two separate pathways for transferring material into or out of the interior of vessel 10A. In an exemplary embodiment, dual-port cap 28 is illustrated in FIGS. 2 and 3. In one embodiment, dual-port cap 28 is constructed from stainless steel, such as 316L stainless steel, but other suitable materials may also be used.

Dual-port cap 28 is removably coupled to vessel 10A at aperture 26. Although shown in FIG. 1 as being attached to the headplate 16 of vessel 10A, dual-port cap 28 and corresponding aperture 26 may be positioned at any point in vessel 10A above the surface 24 of material 20, such as on at least one wall 14.

Dual-port cap 28 illustratively includes a first side 29 facing away from the interior 18 of vessel 10A, and a second side 31 opposite the first side 29 facing towards aperture 26 and the interior 18 of vessel 10A.

Dual-port cap 28 is illustratively coupled to vessel 10A through fitting 41 (FIG. 2). In one exemplary embodiment, fitting 41 is a tri-clamp fitting. Tri-clamp fittings are annular fittings having two portions connected by a hinge on one side and a securing nut and bolt on the opposite side. Fitting 41 illustratively includes an upper extension or flange 42 and a lower extension or flange 43 (see FIG. 3). Fitting 41 is positioned around a portion of dual-port cap 28 and a portion of vessel 10A, and the securing nut and bolt are fastened. Upper flange 42 of fitting 41 is secured against an upper surface 39 of dual-port cap 28, while lower flange 43 is secured against a corresponding extending flange or surface of vessel 10A extending around aperture 26. In one embodiment, fitting 41 provides a fluid-tight seal between dual-port cap 28 and vessel 10A.

In another embodiment (not shown), fitting 41 includes a threaded surface which cooperates with a threaded surface on vessel 10A surrounding aperture 26 (not shown). Tightening the threaded surface of fitting 41 on to the threaded surface of vessel 10A creates a fluid-tight seal between dual-port cap 28 and the interior 18 of vessel 10A. In some embodiments, dual-port cap 28, fitting 41, and/or vessel 10A may include one or more O-rings, gaskets, or other suitable sealing elements to provide the fluid-tight seal between dual-port cap 28 and vessel 10A. Other suitable methods of coupling dual-port cap 28 to vessel 10A may also be used.

Referring again to the exemplary embodiment illustrated in FIG. 1, dual-port cap 28 includes a first connection 30 from first side 29 to second side 31 of dual-port cap 28. First connection 30 fluidly connects first external line 32 to dip tube 34. Dip tube 34 is illustratively a hollow tube extending from second side 31 of first connection 30 through the interior 18 of vessel 10A below the surface 24 of material 20. In an exemplary embodiment, the dip tube 34 is constructed from a ⅜ inch stainless steel tubing, although other suitable sizes and materials may also be used.

Referring next to FIGS. 2 and 3, first external line 32 is fluidly connected to dip tube 34 in first connection 30. First connection 30 is illustratively a fluid-tight connection to prevent fluid from entering headspace 22 of vessel 10A. First connection 30 forms a first fluid passageway extending through dip tube 34, first connection 30, and first external line 32. A portion of material 20 can be removed from the interior 18 of vessel 10A by withdrawing the portion of material 20 through the dip tube 34 and first connection 30 to first external line 32. Steam, such as from steam source 44, or water condensed from such steam, may enter the interior 18 of vessel 10A from first external line 32 through first connection 30 and dip tube 34.

Referring again to the exemplary embodiment illustrated in FIGS. 1-3, dual-port cap 28 includes a second connection 36. Second connection 36 fluidly connects second external line 38 through aperture 26 to the headspace 22 of vessel 10A. Second connection 36 forms a second fluid passageway from second external line 38 through second connection 36 into headspace 22 of vessel 10A. Second connection 36 is illustratively a fluid-tight connection.

A sample may be deposited in the interior 18 of a vessel, such as in the headspace 22 of second vessel 10B, through a second connection 36. Steam, such as from steam source 44, or water condensed from such steam, may enter the interior 18 of vessel 10A from second external line 38 through the second connection 36.

In the exemplary embodiment illustrated in FIGS. 2 and 3, dual-port cap 28 includes recess 46 surrounding dip tube 34. In one embodiment, recess 46 is in fluid communication with headspace 22. Material from second external line 38 enters recess 46 through second connection 36, where it can then proceed into the interior 18 of vessel 10A. Although the illustrated embodiment includes a single connection between second external line 38 and recess 46, in other embodiments, second connection 36 includes two or more connections between second external line 38 and recess 46 surrounding dip tube 34. In some embodiments (not shown), second connection 36 may include one or more diverters or distributors, such as baffles, to more evenly distribute material around dip tube 34.

In one exemplary embodiment, a portion of the second fluid passageway, illustratively the recess 46 in FIGS. 2 and 3, encircles or surrounds a portion of the first fluid passageway, illustratively the dip tube 34. In one exemplary embodiment, the portion of the second fluid passageway that surrounds the portion of the first fluid passageway is coaxial with the portion of the first fluid passageway that it surrounds. In one exemplary embodiment, at least a portion of the second connection 36 is coaxial with the portion of first connection 30 that it surrounds. In one exemplary embodiment, the recess 46 is coaxial with the dip tube 34.

Referring again to FIG. 1, flow of steam or other material through first external line 32 and second external line 38 is illustratively controlled by first valve 48 and second valve 50, respectively. In an exemplary embodiment, first and second valves 48, 50 may be pneumatically controlled valves, such as ¼ inch Aqusyn model SFV1 equipped fail-closed, air-to-open valves. The open or closed status of first and second valves 48, 50 is illustratively controlled by controller 52 through control lines 54 and 56, respectively. In an open status, the valve 48, 50 permits material to pass through the valve 48, 50. In a closed status, the valve 48, 50 prevents material from passing through the valve 48, 50.

In one embodiment, controller 52 controls the status of first and second valves 48, 50 through the selective application of compressed air or gas through control lines 54, 56. In one embodiment, controller 52 includes a programmable logic device, such as a computer, a processor and memory.

First external line 32 and second external line 38 are illustratively connected to form common external line 58 connected to a subheader 60. Common external line 58 may include one or more valves 62 to fluidly isolate a portion of the line. For example, valve 62A controls the flow of material between subheader 60 and the vessel 10A. Vessel 10A is illustratively attached to a steam source 40 for sterilization of the interior 18 vessel 10A, dual-port cap 28, common external line 58, and/or first and second external lines 32, 38. Steam source 40 may be attached to one or more valves 62 to fluidly isolate a portion of the line, and may further include a steam trap 64. In an exemplary embodiment, each valve 62 may be a pneumatically controlled valve, and one or more valves 62 may be operatively coupled to controller 52 or another suitable controller.

In the exemplary embodiment illustrated in FIG. 1, the dual-port cap 28, dip tube 34, first and second external lines 32, 38, and first and second valves 48, 50 (collectively modular system 68), are attached to common external line 58, dip tube 34, by a removable connection 66. By disengaging removable connection 66 and uncoupling dual-port cap 28 from vessel 10A, the modular system 68 can be detached from the vessel 10A and subheader 60. Modular system 68 can then be attached to a second vessel (such as vessel 10B shown in FIG. 5), and replaced with a simplified connection 70 (see FIG. 5) on vessel 10A. In this way, multiple vessels 10 (such as 10A, 10B, etc.) can function as a donor tank and provide a portion of material 20 to a recipient tank, such as vessel 10B using a single modular system 68.

In one embodiment, vessel 10A includes the ability to apply a pressure to the headspace 22 of vessel 10A. In the exemplary embodiment illustrated in FIG. 1, the pressure is illustratively applied by a fluid connection 72 to a pressurized gas source 74. In one embodiment, pressurized gas source 74 is a source of sterile air. In one embodiment, fluid connection 72 fluidly connects to the headspace 22 of the interior 18 of vessel 10A. Although shown as connecting through the headplate 16 of vessel 10A, pressured gas source 74 may be connected to vessel 10A at any point, such as on at least one wall 14. Flow of gas from pressurized gas source 74 though fluid connection 72 is illustratively controlled by gas valve 76. Gas valve 76 may be a pneumatically controlled valve, and may be operatively coupled to controller 52 or another suitable controller.

In one embodiment, vessel 10A includes a fluid connection 72 to a vent 82. Although shown as connecting through the headplate 16 of vessel 10A, vent 82 may be connected to vessel 10A at any point above the surface 24 of material 20, such as on at least one wall 14. Fluid connection between the headspace 22 and vent 82 is illustratively controlled by vent valve 75. Vent valve 75 may be a pneumatically controlled valve, and may be operatively coupled to controller 52 or another suitable controller.

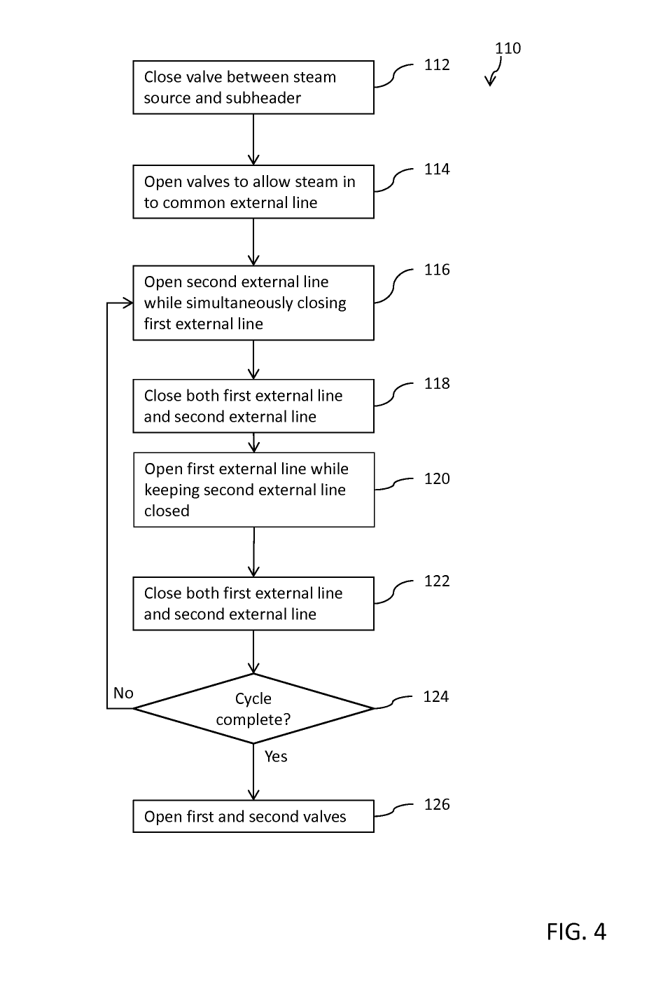

Referring next to FIGS. 1 and 4, an exemplary sterilization procedure 110 for the modular system 68 and vessel 10A is illustrated. Although illustrated a series of blocks 112-122, each block may be performed as a separate step, or one or more blocks may be combined into a single step. In some embodiments, the order of the sterilization procedure 110 may differ from that shown in FIG. 4.

Valve 62A is closed, preventing steam from moving into the subheader 60, as represented in block 112. As shown in block 114, the valves 62 between the steam source 40 and the removable connection 66 are opened to allow steam to enter the common external line 58. The presence of the steam sterilizes the common external line 58.

In blocks 116-120, the controller 52 toggles between opening and closing the first valve 48 and second valve 50. First, as shown in block 116, the first valve 48 is opened while the second valve 50 is closed. This forms an open fluid passageway for the steam to pass through the first connection 30 and in to the dip tube 34. In one embodiment, the first valve 48 is held only for a relatively short period of time as shown in block 116 to prevent too much excess steam from entering the interior 18 of vessel 10A through dip tube 34. Too much excess steam may raise the temperature of the material 20 in vessel 10A, or the water condensing from the steam may dilute the concentration of the material 20. Next, as shown in block 118, both the first valve 48 and second valve 50 are closed. Next, as shown in block 12, the first valve 48 remains closed while the second valve 50 is opened. This forms an open fluid passageway for the steam to pass through the second connection 36 and in to the recess 46 and headspace 22 of vessel 10A.

The exact length of the valve open and close times illustrated in blocks 116, 118, and 120 depends upon many factors, including the length of tubing being sterilized, the size of tubing, the position of the dip tube 34 in the vessel 10A, and the amount of material 20 in the vessel 10A. In one embodiment, valves 48 and 50 are opened and closed to maintain external lines 32 and 38 to a temperature above 121° C. for at least 30 minutes, while accumulating only a small amount of condensate or heat in vessel 10A.

In an exemplary embodiment, valve 48 is held open as shown in block 116 is held for about 5 seconds, both valves are closed as shown in block 118 for 25 seconds, valve 50 is held open as shown in block 120 for about 5 seconds, and both valves are closed as shown in block 122 for 25 seconds.

As shown in block 124, the method then returns to block 116 for the duration of the sterilization cycle. In one exemplary embodiment, the sterilization is about 30 minutes in duration. In another exemplary embodiment, the sterilization cycle is about 60 minutes in duration.

As shown in block 126, the first valve 48 and the second valve 50 are both placed in the open position, if they are not already, to allow any condensed water in the common external line 58, first external line 32, and second external line 38 to drain in to the interior 18 of vessel 10A through dual-port cap 28. This prevents water from being left in the lines.

In one embodiment, the valve 62 between the connection to the steam source and the removable connection and valve 62A are opened during the sterilization procedure 110. Positive pressure from the headspace 22 of vessel 10A through the second connection 36 and second external line 38 sterilizes the common external line 58 and subheader 60 prior to use.

Referring next to FIG. 5, vessel 10A may be fluidly coupled to another vessel 10B to allow for transfer of material 20, such as inoculum or cellular culture, from vessel 10A to vessel 10B. Vessel 10B is similar to vessel 10A, and similar parts are indicated with similar part numbers. Exemplary fluid connections include pipes, tubes, tubing, hoses, conduits, channels, and other suitable connections. In the exemplary embodiment illustrated in FIG. 5, vessel 10A includes modular system 68, including dual-port cap 28 and dip tube 34 extending into the material 20 in the interior 18 of vessel 10A, and vessel 10B includes simplified connection 70 and simplified cap 71. Simplified connection 70 is fluidly connected to the headspace 22 of the interior 18 of vessel 10B through simplified cap 71. In one embodiment, vessel 10B does not include any material 20 in the interior 18, and the interior 18 is completely take up by the headspace 22. In the embodiment illustrated in FIG. 5, vessel 10B includes a material 20, such as water containing nutrients or cellular culture media, and simplified cap provides a fluid-tight fit between simplified connection 70 and vessel 10B.

In another embodiment (not shown), vessel 10B includes a modular system 68 rather than simplified connection 70. To transfer material between vessel 10A and vessel 10B, the first valve 48 associated with the modular system 68 of vessel 10B is closed and the second valve 50 is opened. The second external line 38 attached to the second connection 36 of vessel 10B is fluidly connected to the headspace 18 of vessel 10B. With the first valve 48 closed and the second valve 50 open the modular system 68 associated with vessel 10B then functions as the simplified connection 70.

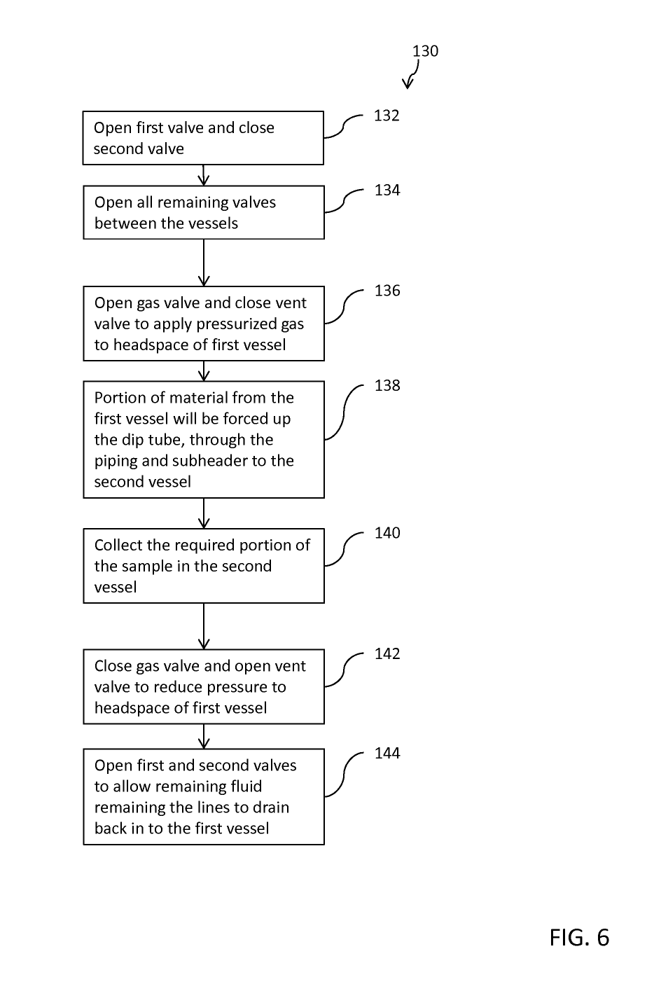

Referring next to FIGS. 5 and 6, and exemplary transfer method 130 for transferring flowable material from vessel 10A to vessel 10B using the dual-port cap 28 is illustrated. Although illustrated as a series of blocks 132-144, each block may be performed as a separate step, or one or more blocks may be combined into a single step. In some embodiments, the order of the transfer method 130 may differ from that shown in FIG. 6.

In one embodiment, the transfer method 130 is performed following the exemplary sterilization procedure 110. In this embodiment, valve 50 remains open from the end of sterilization procedure 110 until immediately prior to the initiation of transfer method 130. This maintains sterility by positive pressure from the headspace 18 of vessel 10A as fluidly connected to common external line 58 and inoculum subheader 60 through second connection 36.

First valve 48 is opened by controller 52, as shown in block 132. As shown in block 134, all remaining valves between the removable connection 66 of vessel 10A and vessel 10B are opened, such as valves 62A, 62B, and any other closed valve. Valve 50 is illustratively closed in block 132 to create a differential pressure to initiate fluid flow through dip tube 34 and first external line 32.

As shown in block 136, a pressurized gas is applied to the headspace 22 of vessel 10A. In one exemplary embodiment, the pressurized gas is sterile air at about 30 psi.

The pressure of the gas in the headspace 22 of vessel 10A forces a portion of material 20 into the dip tube 34, and up in to the first connection 30 of dual-port cap 28, as shown in block 138. The material continues through the common external line 58, through valve 62A in to the subheader 60. The material further continues through valve 62B in to line 77 and through simplified cap 71. The material exits simplified cap 71 through an aperture 26 in vessel 10B and into the headspace 22 in the interior 18 of vessel 10B. As shown in block 140, the pressurized gas is continued to be applied until the desired amount of material has been transferred in vessel 10B. The gas valve 76 is closed, as shown in block 142, and vent valve 75 is opened, removing the pressure on the headspace 22 and stopping the flow of material to vessel 10B.

In one embodiment the gas pressure transfer discussed above is replaced with a pump (not shown) to transfer a portion of material 20 from vessel 10A to vessel 10B. Exemplary pumps include peristaltic pumps. In one embodiment, at least one of first external line 58, inoculum subheader 60, and line 77 includes an in-line mass flow meter (not shown) to determine the amount of inoculum passing through the line. In one embodiment, at least one of vessel 10A and 10B includes an in-tank level probe (not shown) monitoring the current amount of material 24 in the vessel by monitoring the position of surface 24.

The first valve 48 and second valve 50 are set to open, as shown in block 144, if they are not already open. This equilibrates the pressure between the first external line 32 and second external line 38, allowing any material remaining in the first external line 32 or second external line 38 to drain back into vessel 10A through dual-port cap 28.

If vessel 10B also includes a modular system 68 rather than a simplified connection 70, the first and second valves 48 and 50 of that modular system are also set to open, if they are not already, equilibrating the pressure between the lines and allowing any material remaining to drain back into vessel 10B.

In one embodiment, the modular system 68 is designed to be free-draining, such that opening first valve 48 and second valve 50, as shown in block 144, allows the lines to drain relatively clear when material is not being transferred. This reduces the amount of inoculum or cellular culture lost during transfer. By removing at least a portion of the inoculum from the lines, the steam will be better able to access the lines during sterilization, reducing the risk for contamination.

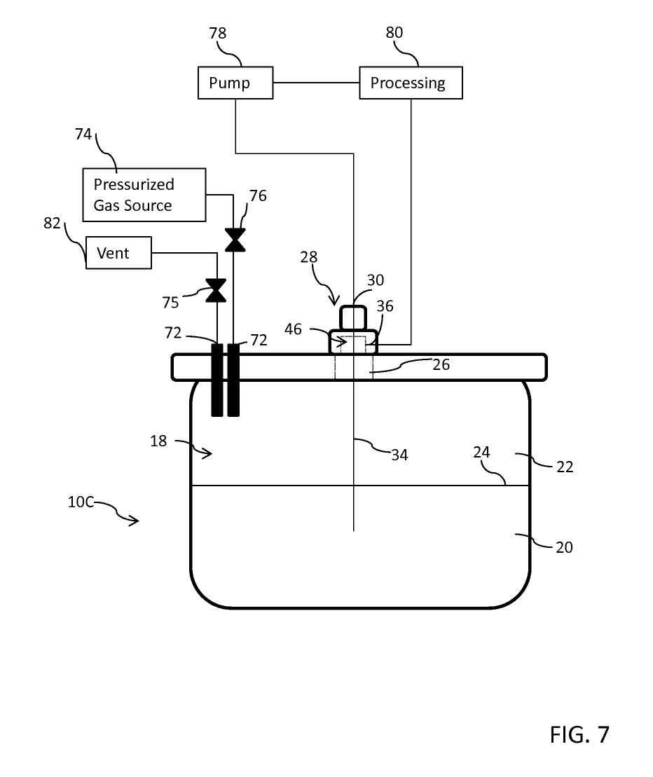

Referring next to FIG. 7, a vessel 10C is illustrated. Vessel 10C has an interior 18, in which a material 20 is positioned. A headspace 22 is present above the surface 24 of the material 20. A dual-port cap 28 is attached to an aperture 26 of vessel 100, and a dip tube 34 extends from the dual-port cap 28 below the surface 24 of the flowable material. In the illustrated embodiment, a portion of the material 20 is drawn up through the dip tube 34 through the first connection 30 of the dual-port cap 28. In one embodiment, the portion of the material 20 is drawn up by pump 78. Although illustrated as being positioned before processing 80, in another embodiment, pump 78 is positioned after processing 80. In another embodiment the portion of the material 20 is drawn up by pressurizing the headspace 22 with gas from a pressurized gas source 74 to processing 80. Following processing 80, the vent valve 76 is opened to reduce the pressure and allow the material to flow from processing 80 back through the second connection 36 into headspace 18 of vessel 10C. A separate vent 82 may be attached to vessel 10C, or processing 80 may include a vent. When the vent is opened, the pressure in the interior 18 of vessel 10C is reduced.

The portion of the material 20 removed from vessel 10C may be subject to processing 80 in one or more processing steps. Exemplary processing steps include filtration, sonication, homogenization, UV treatment, chemical treatment, heating/cooling, mixing, addition of one or more chemicals, sampling, centrifugation, chemical extraction, chromatographic separation, crystallization, precipitation, and other suitable processing steps.

Following the processing by processing 80, the portion of the material 20, which may include all of the material 20, is returned to the interior 18 of vessel 10C through second connection 36 of dual-port cap 28 and into recess 46 surrounding dip tube 34. Recess 46 is fluidly connected to the headspace 22. Thus, the portion of the material 20 being processed leaves and enters the interior 18 of vessel 10C through different connections in the dual-port cap 28, but through a single aperture 26.

While this invention has been described as relative to exemplary designs, the present invention may be further modified within the spirit and scope of this disclosure. Further, this application is intended to cover such departures from the present disclosure as come within known or customary practice in the art to which this invention pertains.

1. A cap for a vessel comprising:
a body having a first side and a second side opposite the first side;
a first fluid passageway extending from the first side to the second side through the cap; and
a second fluid passageway extending from the first side to the second side through the cap, at least a portion of the second fluid passageway surrounding a portion of the first fluid passageway.

2. The cap according to claim 1, wherein the first fluid passageway includes a tube extending from the second side of the cap, the portion of the second fluid passageway surrounding the tube.

3. The cap according to claim 2, wherein the tube is a dip tube configured to extend below a surface of a flowable material in the vessel when the cap is coupled to the vessel.

4. The cap according to claim 3, wherein the cap is configured to withdraw a portion of the flowable material from the vessel through the dip tube and the first fluid passageway, and is further configured to deposit material into a headspace above the surface of the flowable material through the second fluid passageway.

5. The cap according to claim 1, wherein the second fluid passageway includes a recess in the second side, the recess surrounding a portion of the first fluid passageway.

6. The cap according to claim 1, wherein the portion of the second fluid passageway is coaxial with the portion of the first fluid passageway.

7. A vessel for a flowable material comprising:
an interior bounded by a top, a bottom, and at least one wall;
an aperture in the vessel providing access to the interior;
a cap configured to couple to the vessel to cover the aperture, the cap having a first side facing away from the interior of the vessel and a second side facing the interior of the vessel;
a first fluid connection through the cap, the first fluid connection configured to fluidly couple a first external line to the interior of the vessel; and
a second fluid connection through the cap, the second fluid connection configured to fluidly couple a second external line to the interior of the vessel separately from the first fluid connection;
wherein a portion of the second fluid connection surrounds a portion of the first fluid connection.

8. The vessel of claim 7, wherein the cap is removably coupled to the vessel.

9. The vessel of claim 7, wherein the first fluid connection includes a tube extending from the second side of the cap into the interior of the vessel.

10. The vessel of claim 9, wherein the second fluid connection includes a recess in the cap in fluid communication with the interior of the vessel, the recess surrounding the tube.

11. The vessel of claim 10, wherein the recess is coaxial with the tube.

12. The vessel of claim 7, further comprising a first valve coupled to the first fluid connection, the first valve having a closed state preventing the flowable material from being transferred through the first fluid connection and an open state in which the flowable material is permitted to be transferred through the first fluid connection.

13. The vessel of claim 12, further comprising a second valve coupled to the second fluid connection, the second valve having a closed state preventing the flowable material from being transferred through the second fluid connection and an open state in which the flowable material is permitted to be transferred through the second fluid connection.

14. The vessel of claim 13, wherein the first fluid connection includes a tube extending into the interior of the vessel, the vessel further including a pressure source, wherein application of a pressure from the pressure source forces the flowable material to be transferred through the tube and through the first fluid connection and the first valve.

15. A method for sterilizing a vessel, comprising:
providing the vessel, the vessel having an interior bounded by a top, a bottom, and at least one wall, and an aperture providing access the interior;
providing a cap covering the aperture, the cap having a first fluid connection through the cap and a second fluid connection through the cap, at least a portion of the second fluid connection surrounding a portion of the first fluid connection;
fluidly connecting the interior of the vessel to a steam source through the first fluid connection, wherein a flow of steam through the first fluid connection is controlled by a first valve;
fluidly connecting the interior of the vessel to a steam source through the second fluid connection, wherein a flow of steam through the second fluid connection is controlled by a second valve;
opening the first valve to allow steam to sterilize the first fluid connection; and
opening the second valve to allow steam to sterilize the second fluid connection.

16. A method for transferring a flowable material from a first vessel to a second vessel, comprising:
providing the flowable material in an interior of the first vessel, the first vessel including a cap covering an aperture, the cap having a first fluid passageway and a second fluid passageway through the cap, the first fluid passageway including a tube extending from the cap into the flowable material, at least a portion of the second fluid passageway surrounding the tube;
forming a fluid connection between the first vessel and the second vessel by fluidly connecting an exit of the first fluid passageway and the second fluid passageway with the interior of the second vessel;
applying a pressure to the flowable material, the pressure forcing a portion of the flowable material through the tube and first fluid connection;
receiving the portion of the flowable material in the interior of the second vessel.

17. The method of claim 16, wherein the fluid connection between the first fluid passageway and the second vessel includes a first valve having a closed state preventing the flowable material from being transferred through the first fluid passageway and an open state in which the flowable material is permitted to be transferred through the first fluid passageway, and the fluid connection between the second fluid passageway and the second vessel includes a second valve having a closed state preventing the flowable material from being transferred through the second fluid passageway and a second state in which the flowable material is permitted to be transferred through the second fluid passageway.

18. The method of claim 17, further comprising:
opening the first valve prior to applying the pressure to allow the portion of the flowable material to transfer through the first fluid passageway during the application of the pressure, and
opening the second valve after receiving the portion of the flowable material in the interior of the second vessel, wherein opening the second valve allows a second portion of the flowable material in the fluid connection to return to the first vessel.

19. The method of claim 17, wherein the second vessel includes a second cap covering an aperture of the second vessel, the second cap having a first fluid passageway and a second fluid passageway through the second cap, at least a portion of the second fluid passageway of the second cap surrounding a portion of the first fluid passageway of the second cap.

20. The method of claim 19, wherein the portion of the flowable material in the interior of the second vessel is received through the second fluid passageway of the second cap.
